(19) United States
(12) Patent Application Publication
(10) Pub. No.: US 2024/0374578 A1
(43) Pub. Date: Nov. 14, 2024

(54) USE OF MEDICINE FOR REDUCING IRON CONTENT IN BRAIN IN PREPARATION OF DRUG FOR TREATING AND/OR PREVENTING NEUROPSYCHIATRIC DISEASES

(71) Applicant: SOOCHOW UNIVERSITY, Suzhou (CN)

(72) Inventors: Xuechu ZHEN, Suzhou (CN); Pengju Yan, Suzhou (CN); Ningning Li, Suzhou (CN); Shuliu Gao, Suzhou (CN); Zhifeng Shi, Suzhou (CN); Ruichen Gao, Suzhou (CN)

(21) Appl. No.: 18/783,223

(22) Filed: Jul. 24, 2024

Related U.S. Application Data
(63) Continuation of application No. PCT/CN2022/095270, filed on May 26, 2022.

(30) Foreign Application Priority Data
Feb. 17, 2022 (CN) 202210148209.7

(51) Int. Cl. A61K 31/4418; A61K 31/4412; A61P 25/36

(52) U.S. Cl. CPC A61K 31/4418; A61K 31/4412; A61P 25/36

(57) ABSTRACT

The present invention belongs to the technical field of new use of medicines, and provides use of a medicine for reducing the iron content in brain in the preparation of a drug for treating and/or preventing neuropsychiatric diseases. The present invention provides a medicine/diet for reducing the iron content, which prevents the development of substance addiction behavior after administration, to prevent and treat substance addiction or dependence. The medicine is a hypoxia-inducible factor prolyl hydroxylase inhibitor or an iron chelating agent.

### FIELD OF THE INVENTION

The present invention relates to the technical field of new use of medicines, and particularly to use of a medicine for reducing the iron content in brain in the preparation of a drug for treating and/or preventing neuropsychiatric diseases.

### DESCRIPTION OF THE RELATED ART

Drugs refer to natural, semi-synthetic or synthetic substances that can cause dependence, and excite or inhibit the central nervous system, to cause hallucinations, or damages to motor functions, thoughts, behaviors, sensations, and emotions, including opium, morphine, heroin, cocaine, amphetamine, methamphetamine (crystal meth), etc. Drug addiction (substance addiction), also known as drug dependence, drug abuse or substance use disorder, is a chronic recurrent brain disease characterized by compulsive drug seeking and use. Currently, more than 170 countries and regions all over world have drug trafficking problems, and more than 130 countries and regions have drug consumption problems. The number of drug users in the world is more than 300 million, of which more than 35 million are drug addicts. The number of drug-related deaths is increased by 17.5% in the past decade. In China, there were 1.801 million drug addicts by the end of 2020, and 55.49 tons of drugs were seized in 2020. Drug addiction has become a serious social issues, and causes a heavy medical and economic burden to our lives. So far, the main drugs in clinic for abstention from drug abuse include opioid drugs represented by methadone and non-opioid drugs represented by clonidine. Opioid drugs are better at controlling the symptoms, but they are addictive. Although non-opioid drugs are not addictive, they caused obvious withdrawal symptoms and greater side effects. Clinically, there are no particularly effective drugs and clear treatment strategies for substance/drug addiction (especially new synthetic drugs). Therefore, there is an urgent need in clinic to develop a new type of drugs that can prevent drug addiction formation, effectively control withdrawal symptoms, prevent relapse and have less side effects.

Vadadustat (AKB-6548) is a novel oral hypoxia-inducible factor prolyl hydroxylase inhibitor. Many clinical studies have confirmed that Vadadustat can obviously improve renal anemia, and increase the levels of erythropoietin and hemoglobin in patients with chronic kidney disease. Moreover, it could regulate iron metabolism by decreasing hepcidin and increasing transferrin expression, without causing serious adverse reactions. However, the effect of Vadadustat on drug addiction or dependence has not been reported.

Deferiprone (CP20/3-hydroxy-1,2-dimethyl-4-(1H)-pyridone) is an iron chelating agent, which can remove iron ions from ferritin and hemosiderin, and is used to treat diseases caused by acute iron poisoning and chronic iron accumulation. Deferiprone is mainly used in clinic to treat patients with thalassemia who have iron overload and are tolerant to or unwilling to accept current chelating agents treatment. Many studies have reported that Deferiprone can relieve Parkinson's disease and Alzheimer's disease related to iron deposition, and iron overload-induced oxidative stress damage by complexing excessive iron. However, the effect of Deferiprone on drug addiction or dependence has not been reported.

### SUMMARY OF THE INVENTION

To solve the above technical problems, the present invention provides use of a medicine for reducing the iron content in brain in the preparation of a drug for treating and/or preventing neuropsychiatric diseases. The medicine for reducing the brain iron content is selected from Vadadustat and Deferiprone, which indicates the direction for preventing and/or treating substance addiction. The objective of the present invention is to provide a strategy for preventing and treating substance addiction by reducing the brain iron content by a medicine or diet.

In view of drug deficiency in clinical in preventing and treating drug addiction, the present invention provides new use of a medicine or diet for reducing the brain iron content in preventing or treating substance addiction or dependence. Particularly, the present invention provides use of Vadadustat, Deferiprone and low-iron diet in the preparation of a drug for treating and preventing substance addiction or dependence. So far, there is no relevant report on the prevention and treatment of substance addiction or dependence by regulating the brain iron content.

The invention provides use of a medicine for reducing the iron content in brain in the preparation of a drug for preventing and/or treating psychiatric diseases. The medicine can prevent and treat substance addiction or dependence by reducing the iron content in brain.

In an embodiment of the present invention, the psychiatric diseases are caused by substance addiction or dependence.

In an embodiment of the present invention, the substance is morphine or methamphetamine.

In an embodiment of the present invention, the medicine is a hypoxia-inducible factor prolyl hydroxylase inhibitor or an iron chelating agent.

In an embodiment of the present invention, the hypoxia-inducible factor prolyl hydroxylase inhibitor is a compound of Formula I or a pharmaceutically acceptable salt thereof, in which the structure of Formula I is:

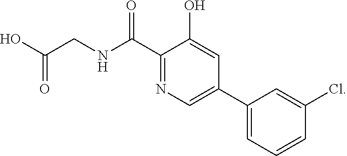 [SMILES: O=C(O)CNC(=O)c1ncc(-c2cccc(Cl)c2)cc1O]

In an embodiment of the present invention, the iron chelating agent is a compound of Formula II or a pharmaceutically acceptable salt thereof, in which the structure of Formula II is:

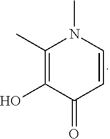

In an embodiment of the present invention, the pharmaceutically acceptable salt is selected from the group consisting of an inorganic acid salt, an organic acid, an alkylsulfonate, an aryl sulfonate and any combination thereof.

In an embodiment of the present invention, the drug for preventing and/or treating psychiatric diseases further includes a pharmaceutically acceptable carrier.

In an embodiment of the present invention, the carrier is selected from the group consisting of a disintegrant, a diluent, a lubricant, a binder, a wetting agent, a flavoring agent, a suspending agent, a surfactant, a preservative and any combination thereof.

In an embodiment of the present invention, the drug for preventing and/or treating psychiatric diseases is in a dosage form of tablets, capsules, soft capsules, granules, pills, oral liquids, emulsions, dry suspensions, dry extracts or injections.

In an embodiment of the present invention, the disintegrant is selected from the group consisting of corn starch, potato starch, cross-linked polyvinylpyrrolidone, sodium carboxymethyl starch, low-substituted hydroxypropyl cellulose, croscarmellose sodium, carboxymethyl cellulose, calcium carboxymethyl cellulose, alginic acid and any combination thereof.

In an embodiment of the present invention, the lubricant is selected from the group consisting of micro-silica powder, magnesium stearate, calcium stearate, stearic acid, talcum powder, anhydrous silica gel and any combination thereof.

In an embodiment of the present invention, the binder is selected from the group consisting of Arabic gum, gelatin, dextrin, hydroxypropyl cellulose, methyl cellulose and polyvinylpyrrolidone, and the wetting agent is selected from sodium dodecyl sulfate, and the flavoring agent can be selected from the group consisting of aspartame, stevioside, sucrose, maltitol, citric acid and any combination thereof.

In an embodiment of the present invention, the suspending agent is selected from the group consisting of Arabic gum, gelatin, methyl cellulose, sodium carboxymethyl cellulose, hydroxymethyl cellulose and aluminum stearate gel, the surfactant is selected from the group consisting of lecithin, sorbitan monooleate and glyceryl monostearate, and the preservative is selected from methyl paraben or/and propyl paraben.

In the present invention, a mouse brain-iron deficiency model is established by feeding mice with low-iron diet for consecutive 3-4 weeks, and then the mice were received intraperitoneal injection of methamphetamine (2 mg/kg/day) to conduct methamphetamine conditioned place preference training for 4 days to build the substance addiction model. After the training, the preference score of the mice residing at the methamphetamine-paired side is measured. The result shows that the preference score of the mice on methamphetamine-paired side in the low-iron diet feeding group is significantly lower than that in the normal diet control group.

In the present invention, a mouse brain-iron deficiency model is established by feeding mice with low-iron diet for consecutive 3-4 weeks, and then the mice were received intraperitoneal injection of morphine (20 mg/kg/day) to conduct morphine conditioned place preference training for 7 days to build the substance addiction model. After the training, the preference score of the mice residing at the morphine-paired side is measured. The result shows that the preference score of the mice in morphine-paired side in the low-iron feed feeding group is significantly lower than that in the normal diet control group.

In the present invention, the mice are given Vadadustat (10 mg/kg/day) by intraperitoneal injection every day, and after 6 hours, the mice were received intraperitoneal injection of morphine (20 mg/kg/day) to conduct morphine conditioned place preference training for 7 days to build the substance addiction model. After the training, the preference score of the mice residing at the morphine-paired side is measured. The result shows that the administration of Vadadustat significantly reduced the preference score of the mice on the morphine-paired side.

In the present invention, the mice are given 1 μL of 13 mg/mL Deferiprone by intracranial stereotaxic implanted tubes (in dorsal hippocampus), and after 6 hours, the mice were received intraperitoneal injection of morphine (20 mg/kg/day) to conduct morphine conditioned place preference training for 7 days to build the substance addiction model. After the training, the preference score of the mice residing at the morphine-paired side is measured. The result shows that the administration of Deferiprone significantly reduced the preference score of the mice on the morphine-paired side.

Low-iron diet, Deferiprone and Vadadustat reduced the iron content in brain and reduced the conditioned place preference score induced by morphine and methamphetamine. Therefore, medicines or diets that reduce the brain iron content have significant effects in the prevention and treatment of substance addiction or dependence.

Compared with the prior art, the technical solution of the present invention has the following advantages:

(1) In view of drugs deficiency in clinical in preventing and treating substance addiction, existing drugs that reduce the brain iron content, such as Vadadustat, Deferiprone, can prevent the development of morphine and methamphetamine addiction behavior after administration (for example, low iron diet, 10 mg/kg/day of Vadadustat and Deferiprone can significantly reduce the conditioned place preference score induced by morphine and methamphetamine).

(2) There are no available new drugs in clinic for prevention or treatment of substance addiction or dependence (such as methamphetamine), and the existing drugs that reduce brain iron content (Vadadustat and Deferiprone, etc.) have effects on methamphetamine addiction or dependence.

(3) Opioid drugs represented by methadone in clinic are addictive in the treatment of opioid addiction (such as morphine), and improper use will lead to drug dependence. The existing drugs that reduce the brain iron content (Vadadustat and Deferiprone, etc.) are not addictive in the treatment of substance addiction.

(4) Non-opioid drugs represented by clonidine for addiction treatment in clinic cause serious adverse reactions after long-term use. The existing drugs that reduce the iron content (Vadadustat and Deferiprone, etc.) are clinically well tolerated with few adverse reactions, and no serious adverse reactions have been reported under a rational dosage.

### BRIEF DESCRIPTION OF THE DRAWINGS

To make the content of the present invention easier to be clearly understood, the following is a further detailed description of the present invention according to the specific embodiments of the invention combined with the attached drawings, in which:

FIG. 1 shows the effect of low-iron diet on the iron content in the brain of mice in the present invention;

FIG. 2 shows the effect of low-iron diet on methamphetamine addiction in mice in the present invention;

FIG. 3 shows the effect of low-iron diet on morphine addiction in mice in the present invention;

FIG. 4 shows the effect of Vadadustat on morphine addiction in the present invention; and

FIG. 5 shows the effect of Deferiprone on morphine addiction in the present invention.

It should be understood that within the scope of the present invention, the technical features described above and the technical features specifically described below (in the embodiments) in the present invention may be combined with each other to constitute a new or preferred technical solution. For the sake of brevity, these combinations are not described here.

### DETAILED DESCRIPTION OF THE PREFERRED EMBODIMENTS

The present invention is further described below with reference to the accompanying drawings and specific embodiments, so that those skilled in the art can better understand and implement it; however, the embodiments given shall not be regarded as a limitation of the present invention

### Example 1. Regulation Effect of Low Iron Diet on Iron Content in Mice Brain

### 1. Experimental Materials

The C57BL/6J mice used in the present invention were purchased from Shanghai Slack Laboratory Animal Co., Ltd, and the low-iron feed used in the present invention was purchased from Jiangsu Xietong Biology Co., Ltd.

### 2. Experimental Method

### 2.1. Method of Drug Administration in Mice

6 male C57BL/6J mice (aged 8 weeks, weighed 22-25 g) were kept in SPF laboratory animal room and randomly divided into a normal diet control group (3 mice) and a low-iron diet control group (3 mice) after 1 week of adaptive feeding. Mice in the low-iron diet group were continuously fed with low-iron feed for 3-4 weeks, and the mice in the normal diet group were fed with normal diet.

### 2.2. Determination of Iron Content in Mouse Brain

After the mice were fed for 3-4 weeks, the mice brain tissue was removed. The tissue sample was placed in a PTFE nitrolysis inner pot, and soaked overnight in 5 mL of nitric acid. The inner lid was closed, and the stainless steel outer jacket was screwed tightly. The system was transferred to a constant-temperature drying oven, and allowed to stand at 80° C. for 2 h, at 120° C. for 2 h, and then at 160° C. for 4 h. The system was then naturally cooled to room temperature in the oven, the inner lid was opened, and the acid solution was heated to almost dryness. The nitrified solution was pipetted into a 25 mL volumetric flask, and the inner pot and the inner lid were washed three times with a small amount of nitric acid solution (1%). The washing solutions were combined into the volumetric flask and the volume was make up to the scale with 1% nitric acid. The iron content was determined by inductively coupled plasma mass spectrometry (ICP-MS).

### 3. Experimental Results

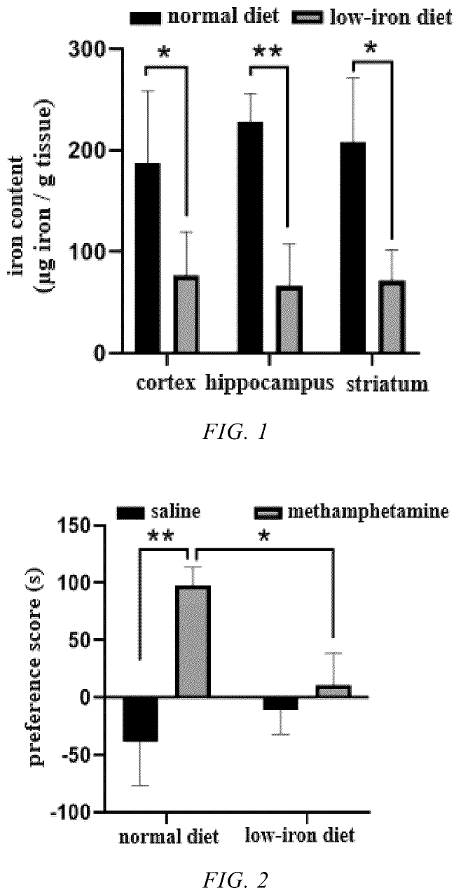

As shown in FIG. 1, the iron content in the striatum, hippocampus, and cortex of mice in the low-iron diet group decrease significantly compared with that in the normal diet group, indicating that low-iron diet can reduce the iron content in brain.

### Example 2. Effect of Low-Iron Diet on Methamphetamine Addiction in Mice

### 1. Experimental Materials

The C57BL/6J mice used in the present invention were purchased from Shanghai Slack Laboratory Animal Co., Ltd, and the low-iron feed used in the present invention was purchased from Jiangsu Xietong Biology Co., Ltd.

### 2. Experimental Method

### 2.1. Method of Administration in Mice

49 male C57BL/6J mice (aged 8 weeks, weighed 22-25 g) were kept in a SPF laboratory animal room. After 1 week of adaptive feeding, the mice were randomly divided into a normal diet+saline control group (11 mice), a normal diet+methamphetamine group (13 mice), a low-iron diet+saline control group (13 mice), and a low-iron diet+methamphetamine group (12 mice). The mice in the low-iron diet group were continuously fed with low-iron feed for 3-4 weeks, and the mice in the normal diet group were fed with normal feed. The mice in the control group were intraperitoneally injected with saline, and the mice in the methamphetamine group were intraperitoneally injected with methamphetamine (2 mg/kg/day), for consecutive 4 days.

### 2.2. Conditioned Place Preference Scoring of Mice

The conditioned place preference boxes (experimental instrument) from Shanghai Jiliang Software Technology Co., Ltd. was used as the behavioral training and testing instrument. The experimental instrument was divided into a drug-paired compartment (black compartment) and a non-drug-paired compartment (white compartment) (the two compartments can be isolated from each other or in communication). On the first day, the mice were allowed to move freely for 900 seconds in the experimental instrument (in communication state), and the activity time of the mice in the drug-paired compartment was recorded and used as the baseline value. On the next day, substance addiction training was carried out. The mice were confined to the drug-paired compartment (in isolated state) after drug administration according to the grouping setting and trained for 60 min/day, for a total of four days. After training, on the sixth day, the mice were allowed to move freely in the experimental instrument (in communication state) for 900 seconds and the activity time of the mice in the drug-paired compartment was recorded. A difference was obtained by subtracting the activity time (baseline) of mice in the drug-paired compartment on the first day from the activity time of mice in the drug-paired compartment on the sixth day, which is the preference score.

### 3. Experimental Results

As shown in FIG. 2, there is a significant difference in the preference scores between the normal diet+saline control group and the normal diet+methamphetamine group, indicating that a 2 mg/kg/day methamphetamine addiction model is successfully established. There is a significant difference in preference scores between the normal diet+methamphetamine group and the low-iron diet+methamphetamine group, indicating that low iron in brain caused by low-iron diet has an effect on methamphetamine addiction.

### Example 3. Effect of Low Iron Diet on Morphine Addiction in Mice

### 1. Experimental Materials

The C57BL/6J mice used in the present invention were purchased from Shanghai Slack Laboratory Animal Co., Ltd, and the low-iron feed used in the present invention was purchased from Jiangsu Xietong Biology Co., Ltd.

### 2. Experimental Method

### 2.1. Method of Administration in Mice

37 male C57BL/6J mice (aged 8 weeks, weighed 22-25 g) were kept in a SPF animal chamber. After 1 week of adaptive feeding, the mice were randomly divided into a normal diet+saline control group (10 mice), a normal diet+morphine group (10 mice), a low-iron diet+saline control group (8 mice), and a low-iron diet+morphine group (9 mice). The mice in the low-iron diet group were continuously fed with low-iron feed for 3-4 weeks, and the mice in the normal diet group were fed with normal feed. The mice in the control group were intraperitoneally injected with saline, and the mice in the morphine group were intraperitoneally injected with morphine (20 mg/kg/day), for consecutive 7 days.

### 2.2. Conditioned Place Preference Scoring of Mice

The conditioned place preference boxes (experimental instrument) from Shanghai Jiliang Software Technology Co., Ltd. was used as the behavioral training and testing instrument. The experimental instrument was divided into a drug-paired compartment (black compartment) and a non-drug-paired compartment (white compartment) (the two compartments can be isolated from each other or in communication). On the first day, the mice were allowed to move freely for 900 seconds in the experimental instrument (in communication state), and the activity time of the mice in the drug-paired compartment was recorded and used as the baseline value. On next day, substance addiction training was carried out. The mice were confined to the drug-paired compartment (in isolated state) after drug administration according to the grouping setting and trained for 30 min/day, for a total of seven days. After training, on the ninth day, the mice were allowed to move freely in the experimental instrument (in communication state) for 900 seconds and the activity time of the mice in the drug-paired compartment was recorded. A difference was obtained by subtracting the activity time (baseline) of mice in the drug-paired compartment on the first day from the activity time of mice in the drug-paired compartment on the ninth day, which is the preference score.

### 3. Experimental Results

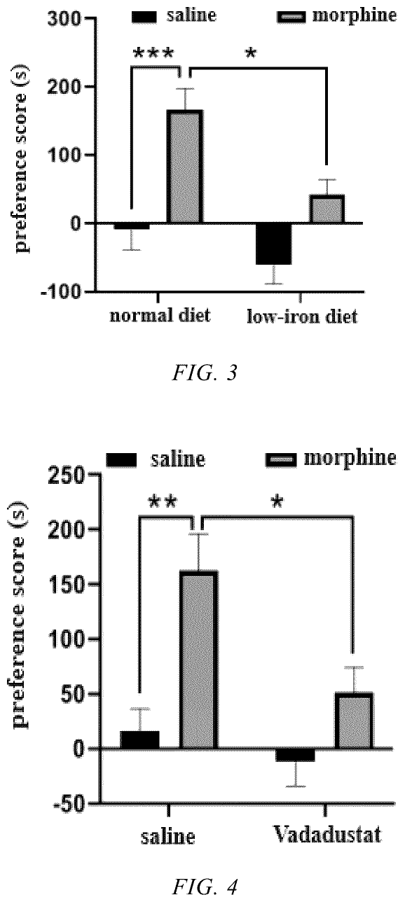

As shown in FIG. 3, there is a significant difference in preference scores between the normal diet+saline control group and the normal diet+morphine group, indicating that a 20 mg/kg/day morphine addiction model is successfully established. There is a significant difference in preference scores between the normal diet+morphine group and the low-iron diet+morphine group, indicating that low iron in brain caused by low-iron diet has an effect on morphine addiction.

### Example 4. Effect of Vadadustat on Morphine Addiction in Mice

### 1. Experimental Materials

The C57BL/6J mice used in the present invention were purchased from Shanghai Slack Laboratory Animal Co., Ltd, and Vadadustat used in the present invention was purchased from the Selleck Corp.

### 2. Experimental Method

### 2.1. Method of Administration in Mice

42 male C57BL/6J mice (aged 8 weeks, weighed 22-25 g) were kept in a SPF laboratory animal room. After 1 week of adaptive feeding, the mice were randomly divided into a saline control group (10 mice), a morphine group (11 mice), a Vadadustat group (10 mice) and a morphine+Vadadustat group (11 mice). The mice in the saline control group were intraperitoneally injected with saline, the mice in the morphine group were intraperitoneally injected with morphine (20 mg/kg/day), and the mice in the morphine+Vadadustat group were intraperitoneally injected with Vadadustat (10 mg/kg/day) and followed by morphine (20 mg/kg/day) injection after 6 h, for consecutive 7 days.

### 2.2. Conditioned Place Preference Scoring of Mice

The conditioned place preference boxes (experimental instrument) from Shanghai Jiliang Software Technology Co., Ltd. were used as the behavioral training and testing instrument. The experimental instrument was divided into a drug-paired compartment (black compartment) and a non-drug-paired compartment (white compartment) (the two compartments can be isolated from each other or in communication). On the first day, the mice were allowed to move freely for 900 seconds in the experimental instrument (in communication state), and the activity time of the mice in the drug-paired compartment was recorded and used as the baseline value. On the next day, substance addiction training was carried out. The mice were confined to the drug-paired compartment (in isolated state) after drug administration according to the grouping setting, and trained for 30 min/day, for a total of seven days. After training, on the ninth day, the mice were allowed to move freely in the experimental instrument (in communication state) for 900 seconds and the activity time of the mice in the drug-paired compartment was recorded. A difference was obtained by subtracting the activity time (baseline) of mice in the drug-paired compartment on the first day from the activity time of mice in the drug-paired compartment on the ninth day, which is the preference score.

### 3. Experimental Results

As shown in FIG. 4, there is a significant difference in preference scores between the saline control group and the morphine group, indicating that a 20 mg/kg/day morphine addiction model is successfully established. There is a significant difference in preference scores between the morphine group and the morphine+Vadadustat group, indicating that Vadadustat of 10 mg/kg/day has an effect on morphine addiction.

### Example 5. Prevention Effect of Deferiprone on Morphine Addiction in Mice

### 1. Experimental Materials

The C57BL/6J mice used in the present invention were purchased from Shanghai Slack Laboratory Animal Co., Ltd, Deferiprone used in the present invention was purchased from Sigma-Aldrich.

### 2. Experimental Method

### 2.1. Method of Administration in Mice

32 male C57BL/6J mice (aged 8 weeks, weighed 22-25 g) were kept in SPF laboratory animal room. After 1 week of adaptive feeding, the mice were randomly divided into a saline control group (8 mice), a morphine group (7 mice), a Deferiprone group (9 mice) and a morphine+Deferiprone group (8 mice). The mice in the saline control group were intraperitoneally injected with saline, the mice in the morphine group were intraperitoneally injected with morphine (20 mg/kg/day), and the mice in the morphine+Deferiprone group was given Deferiprone (13 mg/mL, 1 μL), and then intraperitoneally injected with morphine (20 mg/kg/day) after 6 h, for consecutive 7 days.

### 2.2. Conditioned Place Preference Scoring of Mice

The conditioned place preference boxes (experimental instrument) from Shanghai Jiliang Software Technology Co., Ltd. were used as the behavioral training and testing instrument. The experimental instrument was divided into a drug-paired compartment (black compartment) and a non-drug-paired compartment (white compartment) (the two compartments can be isolated from each other or in communication). On the first day, the mice were allowed to move freely for 900 seconds in the experimental instrument (in communication state), and the activity time of the mice in the drug-paired compartment was recorded and used as the baseline value. On the next day, substance addiction training was carried out. The mice were confined to the drug-paired compartment (in isolated state) after drug administration according to the grouping setting, and trained for 30 min/day, for a total of seven days. After training, on the ninth day, the mice were allowed to move freely in the experimental instrument (in communication state) for 900 seconds and the activity time of the mice in the drug-paired compartment was recorded. A difference was obtained by subtracting the activity time (baseline) of mice in the drug-paired compartment on the first day from the activity time of mice in the drug-paired compartment on the ninth day, which is the preference score.

### 3. Experimental Results

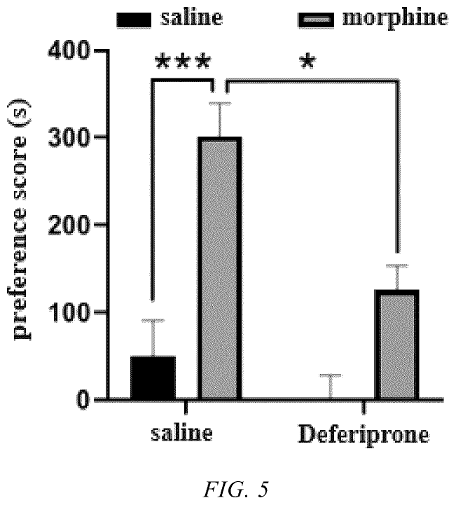

As shown in FIG. 5, there is a significant difference in preference scores between the saline control group and the morphine group, indicating that a 20 mg/kg/day morphine addiction model is successfully established. There is a significant difference in preference scores between the morphine group and the morphine+Deferiprone group, indicating that Deferiprone has an effect on morphine addiction.

It is clear that the above-described embodiments are merely examples provided for clear illustration, and are not intended to limit the implementations of the present invention. Other variations or changes based on the above description can be made by those skilled in the art. The embodiments are not exhaustive herein. Obvious variations or changes derived therefrom also fall within the protection scope of the present invention.

What is claimed is:

1. Use of a medicine for reducing the iron content in brain in the preparation of a drug for preventing and/or treating psychiatric diseases.

2. The use according to claim 1, wherein the psychiatric diseases are caused by substance addiction or dependence.

3. The use according to claim 2, wherein the substance is morphine or methamphetamine.

4. The use according to claim 1, wherein the medicine is a hypoxia-inducible factor prolyl hydroxylase inhibitor or an iron chelating agent.

5. The use according to claim 4, wherein the hypoxia-inducible factor prolyl hydroxylase inhibitor is a compound of Formula I or a pharmaceutically acceptable salt thereof, in which the structure of Formula I is:

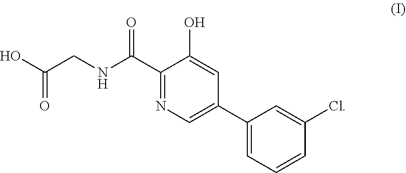 [SMILES: O=C(O)CNC(=O)c1ncc(-c2cccc(Cl)c2)cc1O]

6. The use according to claim 4, wherein the iron chelating agent is a compound of Formula II or a pharmaceutically acceptable salt thereof, in which the structure of Formula II is:

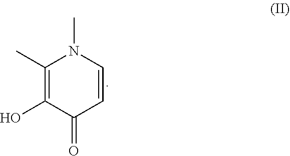

7. The use according to claim 6, wherein the pharmaceutically acceptable salt is selected from the group consisting of an inorganic acid salt, an organic acid salt, an alkylsulfonate, an aryl sulfonate and any combination thereof.

8. The use according to claim 1, wherein the drug for preventing and/or treating psychiatric diseases further comprises a pharmaceutically acceptable carrier.

9. The use according to claim 8, wherein the carrier is selected from the group consisting of a disintegrant, a diluent, a lubricant, a binder, a wetting agent, a flavoring agent, a suspending agent, a surfactant, a preservative and any combination thereof.

10. The use according to claim 1, wherein the drug for preventing and/or treating psychiatric diseases is in a dosage form of tablets, capsules, soft capsules, granules, pills, oral liquids, emulsions, dry suspensions, dry extracts or injections.
